(19) United States
(12) Patent Application Publication
(10) Pub. No.: US 2002/0072061 A1
(43) Pub. Date: Jun. 13, 2002

(54) Methods and compositions for use in synthesizing nucleic acids

(76) Inventors: Alex Chenchik, Palo Alto, CA (US); Karim Syed Hyder, Sunnyvale, CA (US)

Correspondence Address: Bret E. Field, Bozicevic, Field and Francis LLP, Suite 200, 200 Middlefield Road, Menlo Park, CA, 94025, US

(21) Appl. No.: 09/923,761

(22) Filed: Aug. 6, 2001

Related U.S. Application Data
(63) Continuation-in-part of application No. 09/365,122, filed on Jul. 30, 1999, now abandoned.

(51) Int. Cl. C12Q 1/68; C12P 19/34

(52) U.S. Cl. 435/6; 435/91.2

(57) ABSTRACT

The present invention provides methods and compositions for producing a plurality of labeled deoxyribonucleotides from an initial nucleic acid sample. In the subject methods, a solid support bound polyA⁺ RNA fraction is first produced from the initial nucleic acid sample. The resultant solid support bound fraction is then contacted with a plurality of gene specific primers, followed by annealed gene specific primer primed synthesis of a plurality of labeled deoxyribonucleotides. Also provided are kits for use in practicing the subject methods.

### CROSS REFERENCE TO RELATED APPLICATIONS

This application is a continuation-in-part of application Ser. No. 09/1365,122 filed on Jul. 30, 1999; the disclosure of which is herein incorporated by reference

### INTRODUCTION

1. Field of the Invention

The field of this invention is molecular biology, particularly recombinant nucleic acid technology.

2. Background of the Invention

In higher organisms, any given cell expresses only a small fraction of the total number of genes present in its genome. This small fraction of the total number of genes that is expressed determines the life processes carried out by the cell; e.g., development and differentiation, homeostasis, response to insults, cell cycle regulation, aging, apoptosis, and the like. Alterations in gene expression decide the course of normal cell development and the appearance of disease states, such as cancer. Because the choice of which genes are expressed has such a profound effect on the nature of any given cell, methods of analyzing gene expression are of critical import to basic molecular biological research.

Identification of differentially-expressed genes can provide a key to diagnosis, prognosis and treatment of a variety of diseases or condition states in animals, including humans. Additionally, these methods can be used to identify differentially-expressed sequences due to changes in gene expression level associated with predisposition to disease, and the influence of external treatments or infectious agents. Identification of such genes helps in the development of new drugs and diagnostic methods for treating or preventing the occurrence of such diseases.

One way of analyzing gene expression in a particular cell or cell type is to perform differential gene expression assays in which the expression of genes in different cells is compared and any discrepancies in expression are identified. The presence of any discrepancy indicates a difference in the gene(s) expressed in the cells being compared. A method currently employed for high throughput expression profiling and to identify differentially-expressed genes begins with the generation of hybridization probes which are used to probe a set of target nucleic acids, where the target nucleic acids are often arrayed in some manner on a membrane or other solid support.

Despite the promise of analysis of differential gene expression using arrays of target nucleic acids and hybridization probes, there is a continuing need for improvement of the methods currently employed by researchers. For example, in order to detect small changes in gene expression, it is necessary that the background, or non-specific hybridization, be low relative to a true hybridization signal. Background or non-specific hybridization can be decreased substantially if the nucleic acid sample is purified or nucleic acids not involved in differential gene expression are removed from the sample prior to synthesis of the hybridization probes. In addition, preparation of hybridization probes from total RNA is important when using small samples or when looking at rare messages. The present method allows for preparation of hybridization probes from total RNA—not just from polyA⁺ RNA as with other methods known in the art.

The prior art is deficient in highly-specific probe generation from an RNA population in which the resultant product is substantially free of non-specific or background nucleic acids. The present invention fulfills this long-standing need and desire in the art.

### SUMMARY OF THE INVENTION

The technology described herein teaches a method for the generation of hybridization probes from a nucleic acid template, i.e., total RNA, wherein the total RNA or a gene-specific probe is linked to a solid support, either directly or via a ligand. Such hybridization probes are particularly useful when used with nucleic acid arrays. The method minimizes the number of steps and amount of time used for high-throughput screening in clinical labs for diagnostic purposes.

A particular object of the present invention is to provide a method for synthesizing specific nucleic acid probes from a nucleic acid template, i.e., total RNA, using gene-specific primers.

In an embodiment of the present invention, there is provided a method for the preparation of one or more complementary hybridization probes, comprising the steps of: (a) binding a ligand to a solid support; (b) complexing a sample nucleic acid comprising one or more nucleic acid templates with the ligand; (c) combining at least one gene-specific primer, and typically a plurality of gene-specific primer, with the sample nucleic acid, wherein the gene-specific primer(s) anneal to the nucleic acid template(s); (d) initiating synthesis of one or more hybridization probes from the gene-specific primer(s), which are complementary to the nucleic acid template; and (e) removing the complementary hybridization probe(s) from the nucleic acid template.

In yet another embodiment, there is provided a method for the preparation of one or more complementary hybridization probes, comprising the steps of: (a) binding at least one gene-specific primer, and typically a plurality of gene-specific primers, to a solid support; (b) combining a sample nucleic acid comprising one or more nucleic acid templates with the gene-specific primer(s), wherein the gene-specific primer(s) anneal to the nucleic acid template(s); (c) initiating synthesis of a hybridization probe from the gene-specific primer, wherein the hybridization probe is complementary to the nucleic acid template; and (d) removing the nucleic acid template from the complementary hybridization probe.

In still yet another embodiment, there is provided a method for the preparation of one or more complementary hybridization probes, comprising the steps of: (a) binding a sample nucleic acid comprising one or more nucleic acid templates, e.g., mRNAs, to a solid support; (b) combining one or more gene-specific primers with the sample nucleic acid, wherein the gene-specific primer(s) anneal to the nucleic acid template(s); (c) initiating synthesis of one or more hybridization probes from the gene-specific primer(s), wherein the hybridization probe(s) are complementary to the nucleic acid template(s); and (d) removing the complementary hybridization probe(s) from the nucleic acid template.

The methods embodied herein may further comprise the step of incorporating a label into the complementary hybridization probe during synthesis or after synthesis, and may also comprise the step of further amplifying the complementary hybridization probe.

In still yet another embodiment, there is provided a kit for synthesizing complementary hybridization probes from a sample nucleic acid comprising: a solid support; a ligand; and means for attaching the ligand to the support. This embodiment may further comprise a label with which to label the complementary hybridization probes.

Other and further aspects, features, and advantages of the present invention will be apparent from the following description of the preferred embodiments of the invention. These embodiments are given for the purpose of disclosure.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate a preferred embodiment of the invention and should not be considered to limit the scope of the invention.

FIG. 1 is a schematic of the preferred embodiment of the methods of the present invention illustrating various elements, including the solid surface, a ligand (in this case streptavidin/biotin/oligodT), a sample nucleic acid (poly A⁺ RNA) and a gene-specific primer.

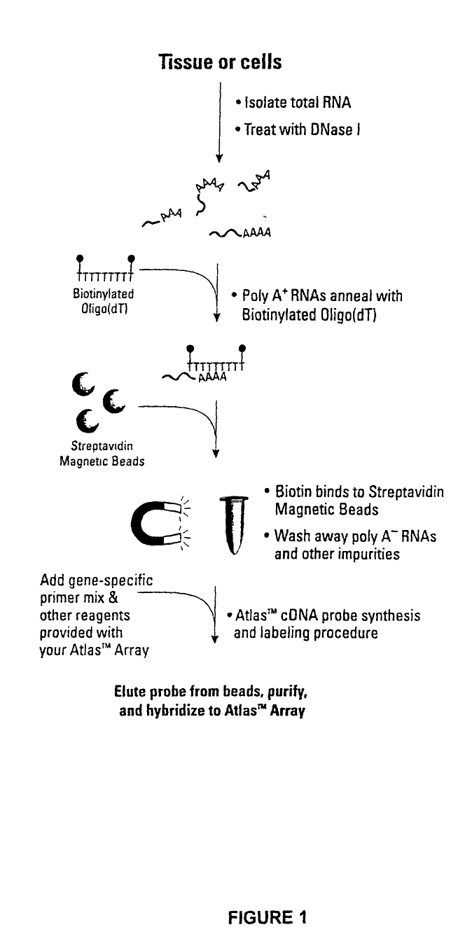

FIG. 2 shows autoradiographies of two Southern blots. Atlas™ Human Array membranes (CLONTECH, Palo Alto, Calif.) were hybridized with 588 gene-specific hybridization probes produced by prior art methods (upper) and the method described herein (lower) using the CDS primer mix from the Atlas™ Human Array Kit (CLONTECH, Palo Alto, Calif.).

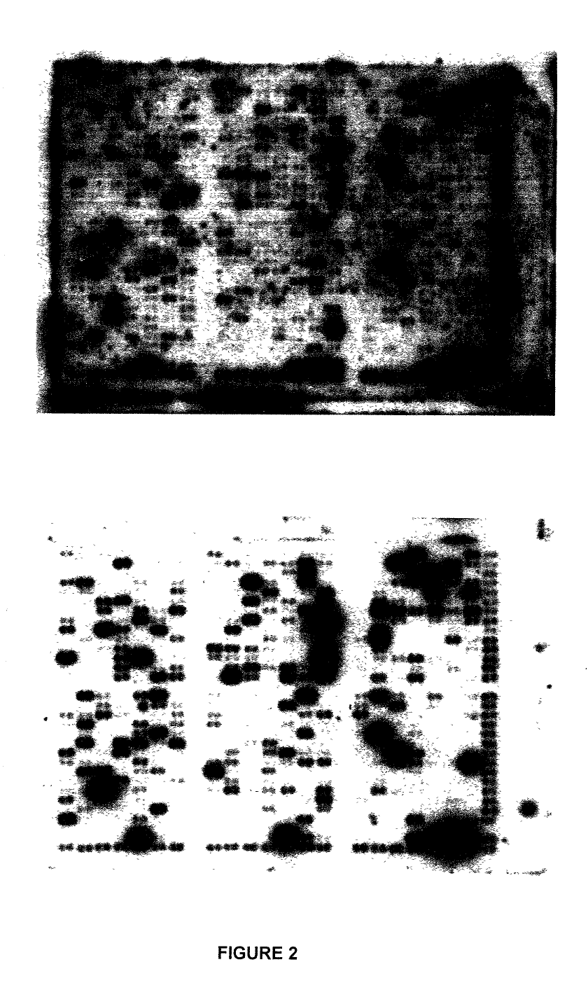

### DETAILED DESCRIPTION OF THE INVENTION

It is an object of the present invention to provide a method for the preparation of one or more complementary hybridization probes, comprising the steps of: (a) binding a ligand to a solid support; (b) complexing a sample nucleic acid comprising one or more nucleic acid templates with the ligand; (c) combining at least one gene-specific primer, and typically a plurality of gene-specific primers, with the sample nucleic acid, wherein the gene-specific primer(s) anneal to the nucleic acid template(s); (d) initiating synthesis of one or more hybridization probes from the gene-specific primer(s), wherein the hybridization probe(s) are complementary to the nucleic acid template; and (e) removing the complementary hybridization probe(s) from the nucleic acid template. Representative ligands include an oligodT/biotin moiety and a gene-specific primer/biotin moiety.

It is yet another object of the present invention to provide a method for the preparation of one or more complementary hybridization probes, comprising the steps of: (a) binding at least one gene-specific primer, and typically a plurality of gene-specific primers, to a solid support; (b) combining a sample nucleic acid comprising one or more nucleic acid templates with the gene-specific primer(s), wherein the gene-specific primer(s) anneal to the nucleic acid template(s); (c) initiating synthesis of a hybridization probe from the gene-specific primer, wherein the hybridization probe is complementary to the nucleic acid template; and (d) removing the nucleic acid template from the complementary hybridization probe. This method may further comprise: (e) removing said hybridization probe from said solid support, typically by means such as heat denaturation, enzymatic cleavage, restriction endonuclease cleavage, site-specific nuclease cleavage, hydrolysis, post-transcriptional modification or chemical treatment (e.g., formamide, guanidine isothiocyanate, etc.).

It is yet another object of the present invention to provide a method for the preparation of one or more complementary hybridization probes, comprising the steps of: (a) binding a sample nucleic acid comprising one or more nucleic acid templates to a solid support; (b) combining one or more gene-specific primers with the sample nucleic acid, wherein the gene-specific primer(s) anneal to the nucleic acid template(s); (c) initiating synthesis of one or more hybridization probes from the gene-specific primer(s), wherein the hybridization probe(s) are complementary to the nucleic acid template(s); and (d) removing the complementary hybridization probe(s) from the nucleic acid template.

Representative solid supports are reaction vials, membranes, beads and bead-like structures. Preferably, reaction vials are glass vials, polypropylene vials, and plastic vials; representative membranes are nylon membranes and nitrocellulose membranes; and representative beads and bead-like structures are magnetic beads, glass beads, dextran, sephadex, sepharose, and cellulose. Typically, the nucleic acid sample is RNA or DNA, and generally, the source of the RNA is from a cell extract, a tissue extract, purified total RNA, purified mRNA or purified poly A⁺ RNA. Typically, methods of removing the complementary hybridization probe include degrading the sample nucleic acid, denaturing the complementary hybridization probe from the nucleic acid template, and destroying the attachment between the ligand or the gene-specific primer or the sample nucleic acid and the solid support.

The above-described methods may further comprise the step of incorporating a label into the complementary hybridization probe during synthesis or after synthesis. Preferable labels are radionucleoside triphosphates, chemiluminescent-modified nucleoside triphosphates, fluorescent-modified nucleotide triphosphates and chemically-modified nucleoside triphosphates. Additionally, the above-described methods may further comprise the step of amplifying the complementary hybridization probe.

It is yet another object of the present invention to provide a kit for synthesizing complementary hybridization probes from a sample nucleic acid comprising: a solid support; a ligand; and means for attaching the ligand to the support. The kit may further comprise a label with which to label the complementary hybridization probes.

As used herein, the term “sample nucleic acid” refers to nucleic acids (DNA or RNA) isolated or produced from a cell, a cell extract, a tissue extract, etc., and include purified total RNA, purified mRNA or purified poly A⁺ RNA. A portion of the sample nucleic acids serve as the template for synthesizing the hybridization probes of the present invention.

As used herein, the terms “nucleic acid template” or “template” refer to the nucleic acid subset of the sample nucleic acid that are isolated by binding to the support-ligand or support-gene-specific primer complex, and which serve as templates for the synthesis of the hybridization probes of the present invention.

As used herein, the terms “hybridization probe” or “complementary hybridization probe” refer to the nucleic acid molecules synthesized that are complementary to the nucleic acid templates isolated from the sample nucleic acid. Additionally, the hybridization probes can be converted to yet another nucleic acid structure or can be amplified by linear or exponential amplification before being used to probe the target nucleic acids. Examples of this process include primer labeling, RNA polymerase mediated transcription or PCR.

As used herein, the term “support” refers to the surface or support, e.g., reaction vials, membranes and beads or bead-like structures, to which the ligand or gene-specific primer is bound.

As used herein, the term “ligand” refers to the intermediary molecule between the support and the nucleic acid template. The ligand is important as it must allow for specific hybridization (and, thus, isolation) of a full set or subset of nucleic acids (nucleic acid templates) from the sample nucleic acid. For example, if the ligand is a gene-specific primer, only those nucleic acid templates that are complementary to the gene-specific primers will hybridize to the ligand/support complex. Alternatively, if an oligo dT ligand is used, nucleic acid templates having a polyA structure will bind to the ligand/support complex. Alternatively, other chemical entities or groups which bind RNA moieties as opposed to the total nucleic acid fraction may be used.

As used herein, the terms “non-specific nucleic acid” or “background nucleic acid” refer to nucleic acids which are not of interest that are contained in the sample nucleic acid. Examples include genomic DNA, tRNAs, etc.

As used herein, the term “gene-specific primer” refers to an oligonucleotide with a defined sequence, having a length of about 10 bases to about 100 bases. In a preferred embodiment, the gene-specific primers of the present invention have a length of about 15 to 30.

A feature of the subject invention is the use of a set (i.e. pool, mixture, collection) of a representational number of gene specific primers to generate labeled nucleic acids from a sample of nucleic acids, usually ribonucleic acids (RNAs), where the labeled nucleic acids may act as “target” in subsequent hybridization assays. As used herein, the term nucleic acid is used in the broadest sense to refer to any sized multimer of nucleotide monomeric units, including short multimers such as dimers, trimers and the like, as well as longer multimers such as oligonucleotides and polynucleotides, where oligonucleotides generally denotes single stranded nucleotide multimers of from about 10 to 100 nucleotides and up to 200 nucleotides in length, and polynucleotides typically refers to single or double stranded nucleotide monomers of generally greater than 100 nucleotides in length.

As the subject sets comprise a representational number of primers, the total number of different primers in any given set will be only a fraction of the total number of different or distinct RNAs in the sample, where the total number of primers in the set will generally not exceed 80%, usually will not exceed 50% and more usually will not 20% of the total number of distinct RNAs, usually the total number of distinct messenger RNAs (mRNAs), in the sample. Any two given RNAs in a sample will be considered distinct or different if they comprise a stretch of at least 100 nucleotides in length in which the sequence similarity is less then 98%, as determined using the FASTA program (default settings). As the sets of gene specific primers comprise only a representational number of primers, with physiological sources comprising from 5,000 to 50,000 distinct RNAs, the number of different gene specific primers in the set of gene specific primers will typically range from about 20 to 10,000, usually from 50 to 2,000 and more usually from 75 to 1500.

Each of the gene specific primers of the sets described above will be of sufficient length to specifically hybridize to a distinct nucleic acid member of the sample, e.g. RNA or cDNA, where the length of the gene specific primers will usually be at least 8 nt, more usually at least 20 nt and may be as long as 25 nt or longer, but will usually not exceed 50 nt. The gene specific primers will be sufficiently specific to hybridize to complementary template sequence during the generation of labeled nucleic acids under conditions sufficient for first strand cDNA synthesis, which conditions are known by those of skill in the art. The number of mismatches between the gene specific primer sequences and their complementary template sequences to which they hybridize during the generation of labeled nucleic acids in the subject methods will generally not exceed 20%, usually will not exceed 10% and more usually will not exceed 5%, as determined by FASTA (default settings).

Generally, the sets of gene specific primers will comprise primers that correspond to at least 20, usually at least 50 and more usually at least 75 distinct genes as represented by distinct mRNAs in the sample, where the term “distinct” when used to describe genes is as defined above, where any two genes are considered distinct if they comprise a stretch of at least 100 nt in their RNA coding regions in which the sequence similarity does not exceed 98%, as determined by FASTA (default settings).

The gene specific oligonucleotide primers may be synthesized by conventional oligonucleotide chemistry methods, where the nucleotide units may be: (a) solely nucleotides comprising the heterocyclic nitrogenous bases found in naturally occurring DNA and RNA, e.g. adenine, cytosine, guanine, thymine and uracil; (b) solely nucleotide analogs which are capable of base pairing under hybridization conditions in the course of DNA synthesis such that they function as the above nucleotides found in naturally occurring DNA and RNA, where illustrative nucleotide analogs include inosine, xanthine, hypoxanthine, 1,2-diaminopurine and the like; or (c) from combinations of the nucleotides of (a) and nucleotide analogs of (b), where with primers comprising a combination of nucleotides and analogues thereof, the number of nucleotide analogues in the primers will typically be less than 25 and more typically less than 5. The gene specific primers may comprise reporter or hapten groups, usually 1 to 2, which serve to improve hybridization properties and simplify detection procedure.

Depending on the particular point at which the gene specific primers are employed in the generation of the labeled nucleic acids, e.g. during first strand cDNA synthesis or following one or more distinct amplification steps, each gene specific primer may correspond to a particular RNA by being complementary or similar, where similar usually means identical, to the particular RNA. For example, where the gene specific primers are employed in the synthesis of first strand cDNA, the gene specific primers will be complementary to regions of the RNAs to which they correspond.

Each gene specific primer can be complementary to a sequence of nucleotides which is unique in the population of nucleic acids, e.g. mRNAs, with which the primers are contacted, or one or more of the gene specific primers in the set may be complementary to several nucleic acids in a given population, e.g. multiple mRNAs, such that the gene specific primer generates labeled nucleic acid when one or more of set of related nucleic acid species, e.g. species having a conserved region to which the primer corresponds, are present in the sample. Examples of such related nucleic acid species include those comprising: repetitive sequences, such as Alu repeats, A1 repeats and the like; homologous sequences in related members of a gene-family; polyadenylation signals; splicing signals; or arbitrary but conversed sequences.

The gene specific primers of the sets of primers according to the subject invention are typically chosen according to a number of different criteria. In some embodiments of the invention, primers of interest for inclusion in the set include primers corresponding to genes which are typically differentially expressed in different cell types, in disease states, in response to the influence of external agents, factors or infectious agents, and the like. In other embodiments, primers of interest are primers corresponding to genes which are expected to be, or already identified as being, differentially expressed in different cell, tissue or organism types. Preferably, at least 2 different gene functional classes will be represented in the sets of gene specific primers, where the number of different functional classes of genes represented in the primer sets will generally be at least 3, and will usually be at least 5. In other words, the sets of gene specific primers comprise nucleotide sequences complementary to RNA transcripts of at least 2 gene functional classes, usually at least 3 gene functional classes, and more usually at least 5 gene functional classes. Gene functional classes of interest include oncogenes; genes encoding tumor suppressors; genes encoding cell cycle regulators; stress response genes; genes encoding ion channel proteins; genes encoding transport proteins; genes encoding intracellular signal transduction modulator and effector factors; apoptosis related genes; DNA synthesis/recombination/repair genes; genes encoding transcription factors; genes encoding DNA-binding proteins; genes encoding receptors, including receptors for growth factors, chemokines, interleukins, interferons, hormones, neurotransmitters, cell surface antigens, cell adhesion molecules etc.; genes encoding cell-cell communication proteins, such as growth factors, cytokines, chemokines, interleukins, interferons, hormones etc.; and the like. Less preferred are gene specific primers that are subject to formation of strong secondary structures with less than −10 kcal/mol; comprise stretches of homopolymeric regions, usually more than 5 identical nucleotides; comprise more than 3 repetitive sequences; have high, e.g. more than 80%, or low, e.g. less than 30%, GC content etc.

The particular genes represented in the set of gene specific primers will necessarily depend on the nature of physiological source from which the RNAs to be analyzed are derived. For analysis of RNA profiles of eukaryotic physiological sources, the genes to which the gene specific primers correspond will usually be Class II genes which are transcribed into RNAs having 5′ caps, e.g. 7-methyl guanosine or 2,2,7-trimethylguanosine, where Class II genes of particular interest are those transcribed into cytoplasmic mRNA comprising a 7-methyl guanosine 5′ cap and a polyA tail.

Solid support

The support may be a test tube or reaction vessel, and the vessel may be arrayed in a multiple-well format. The support may be comprised of a membrane or similar surface (for example, nylon, glass, polypropylene, plastic, etc.). The surface may consist of one or many beads or bead-like structures (for example, magnetic beads, glass, dextran, sephadex, and other synthetic beads). In addition, means for attaching the ligand to the support is necessary. The means for attachment may be part of the support itself or may be an intermediary molecule. A preferred embodiment of the present invention provides a ligand (biotin/oligo-dT) attached to a support having streptavidin available for binding to the biotin component of the ligand.

Ligand

The ligand is a 10-100 deoxythymidine (dT) nucleotide repeat oligo(dT). Oligo(dT) can be also modified on the sugar phosphate or base moiety, which may improve binding stability or other properties of the ligand. The oligo(dT) size is preferably 18-30 nucleotides. In the preferred method, the 3′ end of an oligo(dT)-containing ligand used in the method is blocked at the 3′ end in a manner such that the oligo(dT) ligand does not act as a primer for an extension reaction. Blockage of the 3′ end may include, but is not limited to, attachment to the support, blockage by dideoxythymidine (ddT), or blockage by biotin in the case of a streptavidin-biotin or other appropriate binding pair. Alternatively, the ligand may consist of the gene specific primers themselves, in which case the 3′ end is not blocked and the gene specific primer ligand does serve as a primer for an extension reaction. In addition, the ligand may contain binding groups like biotin or digoxigenin, etc., at the 5′ end or in internal base positions.

Attachment

The ligand may be attached to the support by a covalent bond in the case of direct attachment of oligo dT or gene-specific primers, or through an intermediary binding pair, for example, a streptavidin-biotin-oligodt complex.

Sample Nucleic Acid

The sample nucleic acid may be a cell or tissue extract. The sample nucleic acid of preference is total RNA purified from cells or tissues by any method, including, but not limited to, a phenol/chloroform extraction, use of a silica-based matrix, and an RNA-binding matrix. The sample nucleic acid also may be poly A⁺ (or mRNA) purified from total RNA, or purified directly from cells or tissues by any method, including but not limited to, oligo-dT cellulose, oligo-dT latex, and oligo-dT magnetic beads.

Isolation of RNA

Specific RNAs can be isolated from the sample nucleic acids using the present invention. From the sample nucleic acid, specific and non-specific poly A⁺ RNA can be isolated and separated from other nucleic acids using an oligo-dT ligand. This is accomplished via the deoxyadenosine tail of the poly A⁺ RNA binding to oligo-dT under appropriate conditions. Alternatively, gene-specific primer ligands can be used, in which case nucleic acids specific to the gene-specific primers will be isolated.

Synthesis of Complementary Hybridization Probes

Complementary hybridization probes may be synthesized in the same reaction vessel as the RNA is isolated. Such synthesis can take place while the sample nucleic acid is bound to the ligand and the ligand is bound to the support. Alternatively, reaction conditions may be such that the sample nucleic acid/hybridization probe complex is no longer associated with the ligand, or the sample nucleic acid/hybridization probe/ligand complex is intact, however the ligand is no longer associated with the support. Synthesis occurs under conditions that support DNA polymerases having reverse transcriptase (RT) activity.

Labeling

Labeling of the complementary hybridization probe may be accomplished by the incorporation of one or more analogs of dNTPs into the complementary hybridization probe during its synthesis with reverse transcriptase. Alternatively, labeling of the complementary hybridization probe may comprise post-synthesis modification of the synthesized complementary hybridization probe. The labeling step can be combined with an amplification step, which may involve RNA polymerase transcription or PCR.

Separation of Complementary Hybridization Probe from the Sample Nucleic Acid

If, after synethesis, the hybridization probe is attached to the solid support, the probe can be purified from the reaction components by a washing procedure. Then, the complementary hybridization probe may be removed from the support by means wherein the hybridization probe/sample nucleic acid complex is denatured and the RNA template is degraded; e.g., treatment with RNase H, RNase activity of reverse transcriptase, alkaline hydrolysis with NaOH, melting the RNA-DNA hybrid with high temperature, or any method which destroys the interaction between the comprised hybridization probe and RNA.

The following examples are given for the purpose of illustrating various embodiments of the invention and are not meant to limit the present invention in any fashion.

### EXPERIMENTAL

### Example 1

### Production of Gene-Specific Complementary Hybridization Probes

Ten (10) micrograms of total RNA from human cerebellar tissue was incubated at 70° C. with biotin-dT₃₀-biotin (oligo(dT) containing a biotin group at the 3′ end and one at the 5′ end) (CLONTECH Laboratories, Palo Alto, Calif.) to specifically bind poly(A)⁺ RNA. Streptavidin-coated magnetic particles were then incubated with the biotin-dT-biotin/polyA⁺ RNA complex to form magnetic particle-streptavidin-biotin-dT-poly(A)⁺ (magnetic bead-polyA⁺) complexes.

After three high salt (150 mM NaCl) washes to remove the remaining poly (A)⁻ fraction, the magnetic bead-poly (A)⁺ complex was mixed with gene-specific primers (10× CDS primer mix from the CLONTECH Atlas™ Human Array Kit, CLONTECH Laboratories, Palo Alto, Calif.) specific for 588 known human genes, and incubated at 65° C. for 2 minutes. Reaction buffer, dNTP mix, ³²P-labeled dATP, DTT and MMLV reverse transcriptase (RT) were then added to begin cDNA synthesis at 50° C. for 30 minutes.

After a 30 minute reaction, the reaction mixture was incubated at 70° C. for 5 minutes to denature and disable the reverse transcriptase, and denature the labeled hybridization probes from the magnetic bead-polyA⁺ complex. The labeled hybridization probes were size-fractionated by CLONTECH Chromaspin-200 (CLONTECH Laboratories, Palo Alto, Calif.) and the smaller sized fractions were discarded. The complex mixture of labeled hybridization probes, specific for each of the 588 genes previously mentioned, was heat-denatured, cooled, then added to a nylon Atlas™ Human Array membrane upon which 200-700 basepair long cDNA fragments complementary to the cDNA of the previously-mentioned 588 genes have been affixed. Before addition of labeled probe, the Atlas™ Human Array membrane was pre-hybridized with sheared salmon sperm DNA in hybridization solution (Express-Hyb™, CLONTECH Laboratories, Palo Alto, Calif.) to block any non-specific binding sites. The labeled hybridization probes were incubated overnight with the membrane in roller bottles at 5 rpm rotation at 68° C. in CLONTECH Express-Hyb™ hybridization solution.

After a 16-hour hybridization at 68° C., the membrane was washed four times with high salt buffer (2× SSC, 1% SDS) at 68° C. for 30 min followed by two low salt washes (0.1× SSC, 0.5% SDS) at 68° C. for 30 min. Only the most specific complementary hybridization probes should remain hybridized to the DNA fragments spotted on the membrane. The membrane was placed in sealed plastic and exposed overnight to high-sensitivity X-ray film.

Overnight exposure revealed an extremely low background to signal ratio and a more reproducible and reliable pattern than can be achieved using the traditional labeling procedure. These results confirm that this method of polyA⁺ isolation and probe labeling was successful in eliminating background-causing agents, e.g., DNA and some of the as yet unidentified biological compounds. These agents typically cause non-specific priming and hybridization on the nylon membrane, inhibition of cDNA synthesis, as well as unreproducible gene expression patterns.

One skilled in the art will appreciate readily that the present invention is well adapted to carry out the objects and obtain the ends and advantages mentioned, as well as those objects, ends and advantages inherent herein. The present example, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method for producing a plurality of labeled deoxyribonucleotides from an initial nucleic acid sample, said method comprising:
(a) selectively attaching polyA⁺ RNAs in said initial nucleic acid sample to a solid support(s) to produce a solid support bound polyA⁺ RNA fraction of said initial nucleic acid sample;
(b) combining a plurality of gene-specific primers with said support bound polyA⁺ RNA fraction to anneal said plurality of gene-specific primers to complementary support bound polyA⁺ RNAs of said support bound polyA⁺ RNA fraction;
(c) initiating synthesis of labeled nucleic acids from said annealed gene-specific primers to produce a population of labeled nucleic acids annealed to said support bound polyA⁺ RNA fraction; and
(d) removing said labeled nucleic acids from said support bound polyA⁺ RNA fraction to produce said plurality of labeled deoxyribonucleotides.

2. The method of claim 1, wherein said selectively attaching step (a) comprises:
(i) contacting said initial nucleic acid sample with an oligo-dT/biotin ligand to produce oligo-dT/biotin ligand/polyA⁺ RNA complexes; and
(ii) capturing said oligo-dT/biotin ligand/polyA⁺ RNA complexes on a strept/avidin comprising solid support to produce said solid support bound polyA⁺ RNA fraction.

3. The method of claim 1, wherein said solid support(s) is selected from the group consisting of reaction vials, membranes, beads and bead-like structures.

4. The method of claim 3, wherein said reaction vials are selected from the group consisting of glass vials, polypropylene vials, and plastic vials .

5. The method of claim 3, wherein said membranes are selected from the group consisting of nylon membranes and nitrocellulose membranes.

6. The method of claim 3, wherein said beads and bead-like structures are selected from the group consisting of magnetic beads, glass beads, dextran, sephadex, sepharose, and cellulose.

7. The method of claim 1, wherein said initial nucleic acid sample is selected from the group consisting of a cell extract and tissue extract.

8. The method of claim 1, wherein said method further comprises contacting said plurality of labeled deoxyribonucleotides with an array of nucleic acid fragments.

9. A kit for synthesizing a plurality of labeled deoxyribonucleotides from an initial nucleic acid sample, said kit comprising:
a solid support(s);
a ligand; and
a plurality of gene specific primers.

10. The kit according to claim 9, wherein said solid support(s) is selected from the group consisting of reaction vials, membranes, beads and bead-like structures.

11. The kit according to claim 10, wherein said reaction vials are selected from the group consisting of glass vials, polypropylene vials, and plastic vials.

12. The kit according to claim 10, wherein said membranes are selected from the group consisting of nylon membranes and nitrocellulose membranes.

13. The kit according to claim 10, wherein said beads and bead-like structures are selected from the group consisting of magnetic beads, glass beads, dextran, sephadex, sepharose, and cellulose.

14. The kit according to claim 9, wherein said ligand is an oligo-dT/biotin ligand.

15. The kit of claim 14, further comprising labeled dNTPs.
